(19) United States
(12) Patent Application Publication
(10) Pub. No.: US 2024/0001403 A1
(43) Pub. Date: Jan. 4, 2024

(54) Capacitive Micromachined Ultrasonic Transducer Having Adjustable Bending Angle, And Method For Manufacturing Same

(71) Applicant: Korea Advanced Institute of Science and Technology, Daejeon (KR)

(72) Inventors: Hyun Joo Lee, Daejeon (KR); Sang Mok Lee, Daejeon (KR); Gichan Yun, Daejeon (KR)

(21) Appl. No.: 18/255,070

(22) PCT Filed: Dec. 2, 2021
(86) PCT No.: PCT/KR2021/018069

§ 371 Date: May 30, 2023

(30) Foreign Application Priority Data
Oct. 19, 2021 (KR) 10-2021-0139283

(51) Int. Cl. B06B 1/02

(52) U.S. Cl. CPC B06B 1/0292

(57) ABSTRACT

Disclosed are a capacitive micromachined ultrasonic transducer having an adjustable bending angle and a method for manufacturing same. The ultrasonic transducer according to one embodiment may comprise: a substrate; a plurality of transducer elements spaced apart from each other and stacked on top of the substrate; flexible hinges which are positioned between the plurality of transducer elements and formed so as to pass through the substrate; a first polymer layer formed so as to cover the bottom of the substrate; and an actuator layer formed under the first polymer layer.

### TECHNICAL FIELD

The following disclosure relates to a capacitive micromachined ultrasonic transducer having an adjustable bending angle and a method of manufacturing the same.

### BACKGROUND ART

A capacitive micromachined ultrasonic transducer (CMUT) has a structure with a membrane positioned above a micromachined cavity and may be used to convert an acoustic signal into an electrical signal or an electrical signal into an acoustic signal.

CMUT is a micromachined device, and a two-dimensional (2D) transducer array may be more easily configured using a CMUT. Therefore, compared to other transducer arrays, a transducer array including a CMUT may include more transducers and provide a wider bandwidth.

Conventional ultrasonic transducers convert signals based on piezoelectricity, but a CMUT performs energy conversion based on a change in the capacitance of a cavity caused by vibrations of a membrane. In general, a CMUT is biased using a direct current (DC) voltage that determines an operating position of a device. When an alternating current (AC) signal is applied to electrodes of the biased CMUT, the membrane vibrates to generate an ultrasonic wave, such that the CMUT operates as an ultrasonic transmitter. On the other hand, when an ultrasonic wave is applied to the membrane of the biased CMUT, an electrical signal is generated as the capacitance of the CMUT changes, such that the CMUT operates as an ultrasonic receiver.

### DISCLOSURE OF THE INVENTION

### Technical Goals

An ultrasonic focusing technology that is reusable and does not require a complicated circuit is demanded.

According to an embodiment, it is possible to provide a technique for causing an ultrasonic beam to be focused by variably controlling the bending angle of an ultrasonic transducer.

However, the technical goals are not limited to those described above, and other technical goals may be present.

### Technical Solutions

An ultrasonic transducer according to an embodiment may include a substrate, a plurality of transducer elements stacked on a top of the substrate to be spaced apart from each other, a flexible hinge positioned between the plurality of transducer elements and formed to pass through the substrate, a first polymer layer formed to cover a lower portion of the substrate, and an actuator layer formed on a bottom of the first polymer layer.

The flexible hinge may include a second polymer layer positioned over a separation space formed between adjacent transducer elements, and a liquid metal layer extending from a bottom of the second polymer layer and passing through the substrate.

The actuator layer may include an insulating layer formed on the bottom of the first polymer layer, a first electrode layer formed on a bottom of the insulating layer, a dielectric elastomer formed on a bottom of the first electrode layer, and a second electrode layer formed on a bottom of the dielectric elastomer.

The first polymer layer may include polydimethylsiloxane.

The second polymer layer may include polyimide.

The liquid metal layer may include a bismuth (Bi)-lead (Pb)-indium (In)-tin (Sn)-cadmium (Cd) fusible alloy.

The liquid metal layer may undergo a phase transition from solid to liquid based on heat generated by a voltage applied to the substrate.

The dielectric elastomer may bend by a voltage applied to the first electrode layer and the second electrode layer when a fusible alloy included in the flexible hinge is in a liquid state.

A method of manufacturing an ultrasonic transducer according to an embodiment may include forming a substrate, stacking a plurality of transducer elements on a top of the substrate to be spaced apart from each other, forming a flexible hinge between the plurality of transducer elements to pass through the substrate, forming a first polymer layer to cover a lower portion of the substrate, and forming an actuator layer on a bottom of the first polymer layer.

The forming of the flexible hinge may include forming a second polymer layer over a separation space formed between adjacent transducer elements, and forming a liquid metal layer extending from a bottom of the second polymer layer and passing through the substrate.

The forming of the second polymer layer may include stacking a polymeric material on the substrate and the plurality of transducer elements, and forming the second polymer layer by patterning the polymeric material.

The forming of the liquid metal layer may include forming a trench by etching the substrate positioned on the bottom of the second polymer layer, and forming the liquid metal layer by filling the trench with a liquid metal.

The forming of the actuator layer may include forming an insulating layer on the bottom of the first polymer layer, forming a first electrode layer on a bottom of the insulating layer, forming a dielectric elastomer on a bottom of the first electrode layer, and forming a second electrode layer on a bottom of the dielectric elastomer.

The first polymer layer may include polydimethylsiloxane.

The stacking of the polymeric material may include stacking polyimide through spin coating.

The liquid metal layer may include a Bi-Pb-In-Sn-Cd fusible alloy.

The liquid metal layer may undergo a phase transition from solid to liquid based on heat generated by a voltage applied to the substrate.

The dielectric elastomer may bend by a voltage applied to the first electrode layer and the second electrode layer when a fusible alloy included in the flexible hinge is in a liquid state.

An ultrasonic transducer system according to an embodiment may include the ultrasonic transducer of claim 1, and a controller configured to control the ultrasonic transducer.

The controller may be further configured to control a flexible hinge included in the ultrasonic transducer and an actuator layer included in the ultrasonic transducer independently of driving the ultrasonic transducer.

### BRIEF DESCRIPTION OF DRAWINGS

FIG. 1A shows a cross-section of an ultrasonic transducer according to an embodiment.

FIG. 1B is a view of the ultrasonic transducer shown in FIG. 1A from above.

FIG. 2 is a view illustrating an operation of controlling a bending angle of the ultrasonic transducer shown in FIG. 1A.

FIG. 3 is a simplified block diagram of an example of an ultrasonic transducer system according to an embodiment.

FIG. 4 is a diagram illustrating the ultrasonic transducer system shown in FIG. 3.

FIG. 5 is a diagram illustrating a radius of curvature (ROC) according to a bending angle of the ultrasonic transducer shown in FIG. 1A.

FIG. 6 illustrates graphs of −3 dB distances according to a bending angle of the ultrasonic transducer shown in FIG. 1A.

FIG. 7 is a table illustrating −3 dB distances according to a bending angle of the ultrasonic transducer shown in FIG. 1A.

FIG. 8 illustrates a graph of a maximum pressure ratio according to a bending angle of the ultrasonic transducer shown in FIG. 1A.

FIG. 9 is a table illustrating a maximum pressure ratio according to a bending angle of the ultrasonic transducer shown in FIG. 1A.

FIG. 10 is a simplified block diagram of another example of an ultrasonic transducer system according to an embodiment.

FIGS. 11A to 11D are views illustrating a method of manufacturing the ultrasonic transducer shown in FIG. 1A.

### BEST MODE FOR CARRYING OUT THE INVENTION

The following detailed structural or functional description is provided as an example only and various alterations and modifications may be made to the embodiments. Here, the embodiments are not construed as limited to the disclosure and should be understood to include all changes, equivalents, and replacements within the idea and the technical scope of the disclosure.

Terms, such as first, second, and the like, may be used herein to describe components. Each of these terminologies is not used to define an essence, order or sequence of a corresponding component but used merely to distinguish the corresponding component from other component(s). For example, a first component may be referred to as a second component, and similarly, the second component may also be referred to as the first component.

It should be noted that if it is described that one component is “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or populations thereof.

Unless otherwise defined, all terms, including technical and scientific terms, used herein have the same meaning as commonly understood by those having ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, embodiments will be described in detail with reference to the accompanying drawings. When describing the embodiments with reference to the accompanying drawings, like reference numerals refer to like elements and a repeated description related thereto will be omitted.

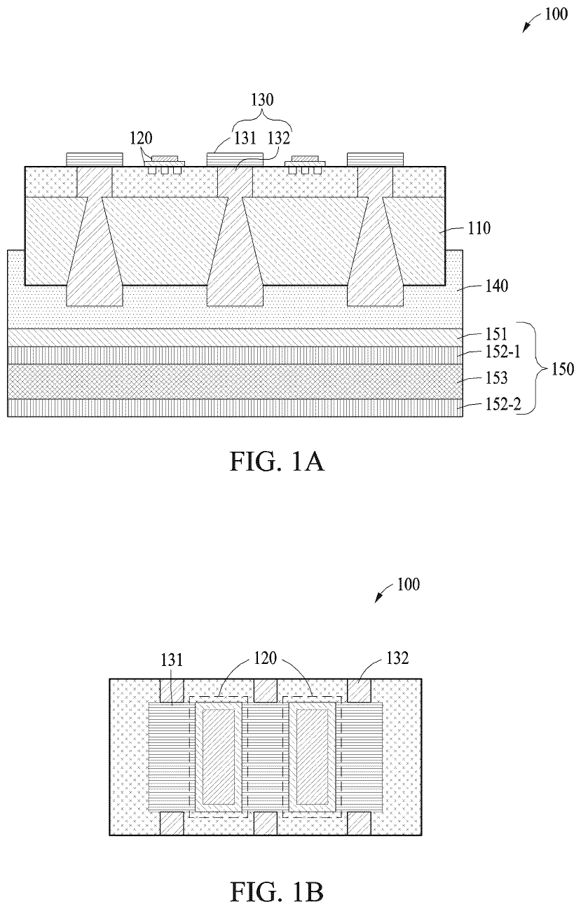

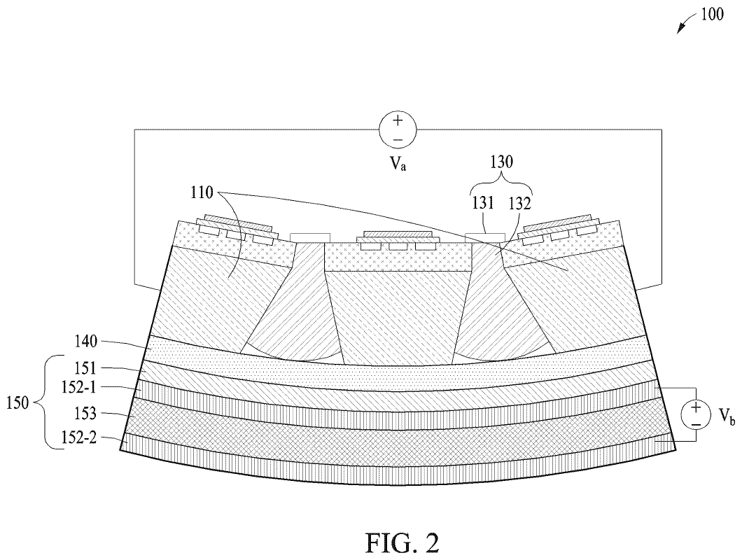

FIG. 1A shows a cross-section of an ultrasonic transducer according to an embodiment, and FIG. 1B is a view of the ultrasonic transducer shown in FIG. 1A from above.

A capacitive ultrasonic transducer 100 may generate an ultrasonic wave by converting electrical energy into mechanical energy. The capacitive ultrasonic transducer 100 may cause the generated ultrasonic beam to be focused. The ultrasonic transducer 100 may bend, and may deform reversibly, such as maintaining the bending shape or deforming back. The ultrasonic transducer 100 may cause the ultrasonic beam to be focused while maintaining the intensity of the ultrasonic beam.

The ultrasonic transducer 100 may include a substrate 110, a plurality of transducer elements 120, a flexible hinge 130, a first polymer layer 140, and an actuator layer 150.

The substrate 110 may be a silicon substrate, and the transducer elements 120 or the like may be stacked thereon.

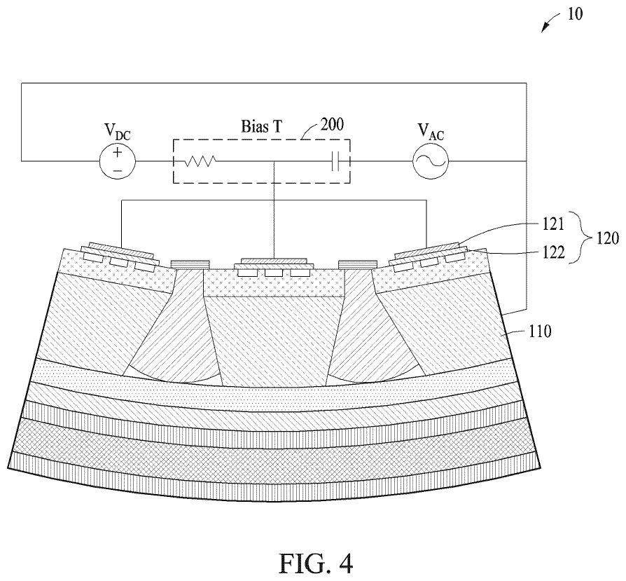

The plurality of transducer elements 120 may be stacked on the top of the substrate 110 to be spaced apart from each other. The plurality of transducer elements 120 may be driven simultaneously to form an ultrasonic beam with strong pressure, and may be driven separately to form various ultrasonic beams. Driving the plurality of transducer elements 120 will be described in detail with reference to FIG. 3.

The flexible hinge 130 may be positioned between the plurality of transducer elements 120 and formed to pass through the substrate 110. The flexible hinge 130 may contribute to the flexible and reversible deformation (e.g., bending) of the capacitive ultrasonic transducer 100. The flexible hinge 130 may include a second polymer layer 131 and a liquid metal layer 132.

The second polymer layer 131 may be positioned over a separation space formed between the transducer elements 120. The second polymer layer 131 may be formed through spin coating of polyimide capable of withstanding the temperature at which a liquid metal included in the liquid metal layer 132 changes into a liquid. The second polymer layer 131 may contribute to the flexible deformation of the ultrasonic transducer 100.

The liquid metal layer 132 may extend from the bottom of the second polymer layer 131 and pass through the substrate 110. The liquid metal layer 132 may include a bismuth (Bi)-lead (Pb)-indium (In)-tin (Sn)-cadmium (Cd) fusible alloy. The Bi-Pb-In-Sn-Cd fusible alloy may be solid at room temperature and may be liquid at 47° C. or higher. The liquid metal layer 132 may undergo a phase transition from solid to liquid based on heat generated by a voltage applied to the substrate 110. The liquid metal layer 132 may contribute to the reversible deformation of the ultrasonic transducer 100.

The first polymer layer 140 may be formed to cover the lower portion of the substrate 110. The first polymer layer 140 may be formed of polydimethylsiloxane, which is a flexible material, to prevent the liquid metal included in the liquid metal layer 132 from leaking out when it changes to liquid.

The actuator layer 150 may be formed on the bottom of the first polymer layer 140. The actuator layer 150 may control the bending angle of the ultrasonic transducer 100, thereby contributing to causing the ultrasonic beam to be focused while maintaining the intensity of the ultrasonic beam. The actuator layer 150 may include an insulating layer 151, a first electrode layer 152-1, a second electrode layer 152-2, and a dielectric elastomer 153.

The insulating layer 151 may be formed on the bottom of the first polymer layer 140. The insulating layer 151 may include a silicon elastomer so that the actuator layer 150 may maintain a state of being electrically insulated from the transducer elements 120.

The first electrode layer 152-1 may be positioned on the top of the dielectric elastomer 153, and the second electrode layer 152-2 may be positioned on the bottom of the dielectric elastomer 153. The first electrode layer 152-1 and the second electrode layer 152-2 may serve as an upper electrode and a lower electrode of the actuator 150, respectively. The first electrode layer 152-1 and the second electrode layer 152-2 may be formed of carbon powder.

The dielectric elastomer 153 may be formed on the bottom of the first electrode layer 152-1. The dielectric elastomer 153 may be formed of an acrylic elastomer that bends when a high voltage is applied thereto. When the fusible alloy included in the flexible hinge 130 is in a liquid state, the dielectric elastomer 153 may bend by a voltage applied to the first electrode layer 152-1 and the second electrode layer 152-2.

The capacitive ultrasonic transducer 100 may bend through the flexible hinge 130, the first polymer layer 140, and the actuator layer 150. Specifically, the capacitive ultrasonic transducer 100 may perform a reversible deformation, such as maintaining the shape or deforming, using a phase transition of the liquid metal included in the flexible hinge 130. In addition, the ultrasonic transducer 100 may control the bending angle through the dielectric elastomer 153 included in the actuator layer 150, thereby causing the ultrasonic beam to be focused while maintaining the intensity of the ultrasonic beam. Hereinafter, the operation of controlling the bending angle of the ultrasonic transducer 100 will be described in detail.

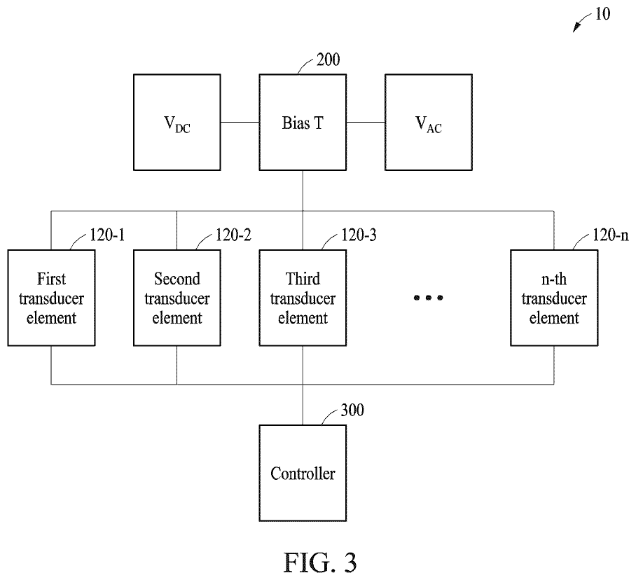

FIG. 2 is a view illustrating an operation of controlling a bending angle of the ultrasonic transducer shown in FIG. 1A.

Referring to FIG. 2, the bending angle of the capacitive ultrasonic transducer 100 may be controlled by applying voltages (e.g., Va and Vb).

When a direct current (DC) voltage Va is applied to both ends of the substrate 110 of the capacitive ultrasonic transducer 100, heat may be generated in the silicon substrate 110 and the liquid metal layer 132. The fusible alloy included in the liquid metal layer 132 may undergo a phase transition from solid to liquid as the voltage Va increases. The Bi-Pb-In-Sn-Cd fusible alloy included in the liquid metal layer 132 may have a characteristic of being solid at room temperature and undergoing a phase transition to liquid at 47° C. or higher.

After the phase transition of the Bi-Pb-In-Sn-Cd fusible alloy to liquid, a DC voltage Vb may be applied to the upper electrode 152-1 and the lower electrode 152-2 of the actuator 150. When the voltage Vb is applied to the upper electrode 152-1 and the lower electrode 152-2, the top surface of the dielectric elastomer 153 (e.g., the surface in contact with the electrode 152-1) may receive a compressive force, and the bottom surface of the dielectric elastomer 153 (e.g., the surface in contact with the electrode 152-2) may receive a tensile force. As different forces are applied respectively to the top surface and the bottom surface of the dielectric elastomer 153, the dielectric elastomer 153 may bend, and the ultrasonic transducer 100 may also bend. The magnitude of the voltage Vb may correspond to the bending angle of the ultrasonic transducer 100, and as the magnitude of the voltage Vb increases, the bending angle of the ultrasonic transducer 100 may increase.

When it is desired to maintain the bending angle of the ultrasonic transducer 100, the temperature of the fusible alloy may be lowered by eliminating the DC voltage Va applied to both ends of the substrate 110. When the temperature of the fusible alloy falls below 47° C., the fusible alloy may undergo a phase transition from liquid to solid and harden. Therefore, the ultrasonic transducer 100 may harden in the bending state, and the bending angle of the ultrasonic transducer 100 may be maintained even when the DC voltage Vb applied to the upper electrode 152-1 and the lower electrode 152-2 of the actuator 150 is eliminated.

When it is desired to change the bending angle of the ultrasonic transducer 100, the intensity of the voltage Vb applied to the upper electrode 152-1 and the lower electrode 152-2 of the actuator 150 may be adjusted in a state where the voltage Va is applied to both ends of the substrate 110, whereby the bending angle of the ultrasonic transducer 100 may be changed.

When it is desired to eliminate the bending angle of the ultrasonic transducer 100 (e.g., when it is desired to return to the flat state), the voltage Va may be applied again to both ends of the substrate 110 to cause a phase transition of the fusible alloy to liquid, rather than applying the voltage Vb to the upper electrode 152-1 and the lower electrode 152-2 of the actuator 150, whereby the bending angle of the ultrasonic transducer 100 may be eliminated. When the voltage Va is eliminated thereafter, the fusible alloy may undergo a phase transition to solid, such that the state where the bending angle of the ultrasonic transducer 100 is eliminated may be maintained.

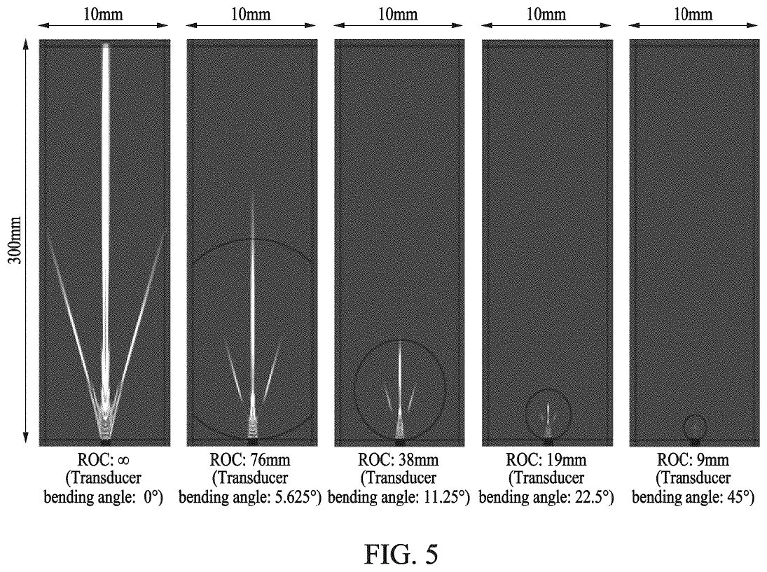

FIG. 3 is a simplified block diagram of an example of an ultrasonic transducer system according to an embodiment, and FIG. 4 is a diagram illustrating the ultrasonic transducer system shown in FIG. 3.

An ultrasonic transducer system 10 may include an ultrasonic transducer (the ultrasonic transducer 100 shown in FIG. 1), a bias T 200, and a controller 300. The ultrasonic transducer system 10 may generate an ultrasonic wave by driving the ultrasonic transducer 100 through the bias T 200 and control the bending angle of the ultrasonic transducer 100 through the controller 300 to cause the ultrasonic wave to be focused.

The ultrasonic transducer 100 may generate an ultrasonic wave as a DC voltage V_{DC} or an AC voltage V_{AC} is applied thereto, and may cause the ultrasonic wave to be focused according to the bending angle.

The DC voltage V_{DC} may be applied to an upper electrode 121 and a lower electrode (e.g., the substrate 110) of a transducer element (the transducer element 120 shown in FIG. 1) to vibrate a membrane 122. Compared to the AC voltage V_{AC}, the DC voltage V_{DC} may cause the membrane 122 to move further toward the lower electrode 110, thereby achieving a higher sound wave efficiency.

The AC voltage V_{AC} may generate an ultrasonic wave in the form of a sine wave or square wave, and may be applied in a unipolar or bipolar form. The AC voltage V_{AC} may be applied corresponding to the unique vibration frequency of the transducer element 120, thereby vibrating the membrane 122 and generating a sound wave.

The Bias T 200 may simultaneously apply the DC voltage V_{DC} or the AC voltage V_{AC} for generating an ultrasonic wave to the plurality of transducer elements 120 included in the ultrasonic transducer 100. The bias T 200 may include a capacitor and a resistor or a capacitor and an inductor.

The controller 300 may control the bending angle of the ultrasonic transducer 100 to cause an ultrasonic beam generated by the ultrasonic transducer 100 to be focused. The controller 300 may independently control the flexible hinge 130 included in the ultrasonic transducer 100 and the actuator layer 150 included in the ultrasonic transducer 100. That is, the controller 300 may control the bending angle of the ultrasonic transducer independently of driving the ultrasonic transducer 100.

The ultrasonic transducer system 10 may obtain ultrasonic beams having various focusing shapes using the ultrasonic transducer 100 that variably bends at various angles, and may cause an ultrasonic beam with a stronger intensity to be focused on a very small local area by bending the ultrasonic transducer 100 more.

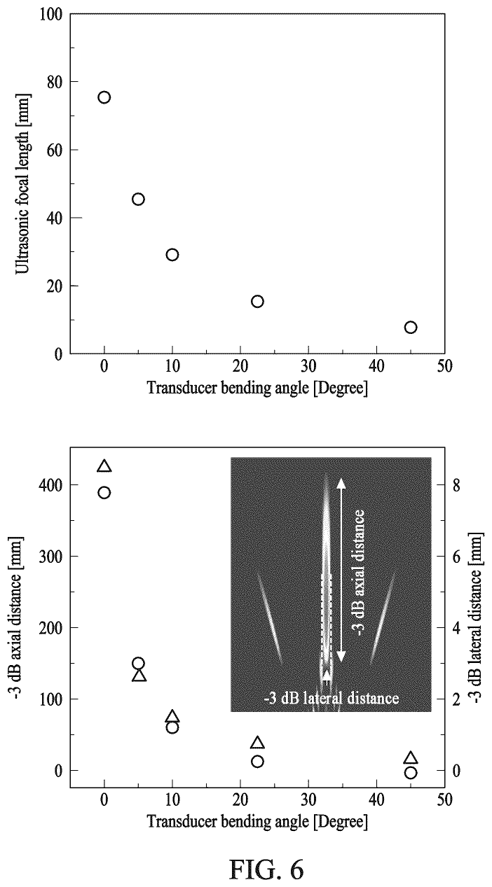

FIG. 5 is a diagram illustrating a radius of curvature (ROC) according to a bending angle of the ultrasonic transducer shown in FIG. 1A.

Referring to FIG. 5, eight transducer elements may be included in an ultrasonic transducer, the distance between transducer elements may be 500 micrometers (um), and a membrane of an ultrasonic transducer may have a thickness of 0.5 nanometers (nm) and vibrate at 5 megahertz (MHz). In addition, an ultrasonic medium may be water, and the size of the medium may be 10 millimeters (mm) wide and 300 mm long.

When the bending angle of the ultrasonic transducer doubles, the radius of curvature (ROC) of the ultrasonic beam may halve. For example, when the bending angle of the ultrasonic transducer is 0°, the ROC of the ultrasonic beam may be infinite, such that the ultrasonic beam may reach the end of the medium. When the bending angle of the ultrasonic transducer is 5.625°, the ROC of the ultrasonic beam may be 76 mm. When the bending angle of the ultrasonic transducer is 11.25°, the ROC of the ultrasonic beam may be 38 mm. When the bending angle of the ultrasonic transducer is 45°, the ROC of the ultrasonic beam may be 9 mm.

That is, as the ultrasonic transducer bends more, the ROC of the ultrasonic beam may decrease more, and the ultrasonic beam may be focused on a more minute area.

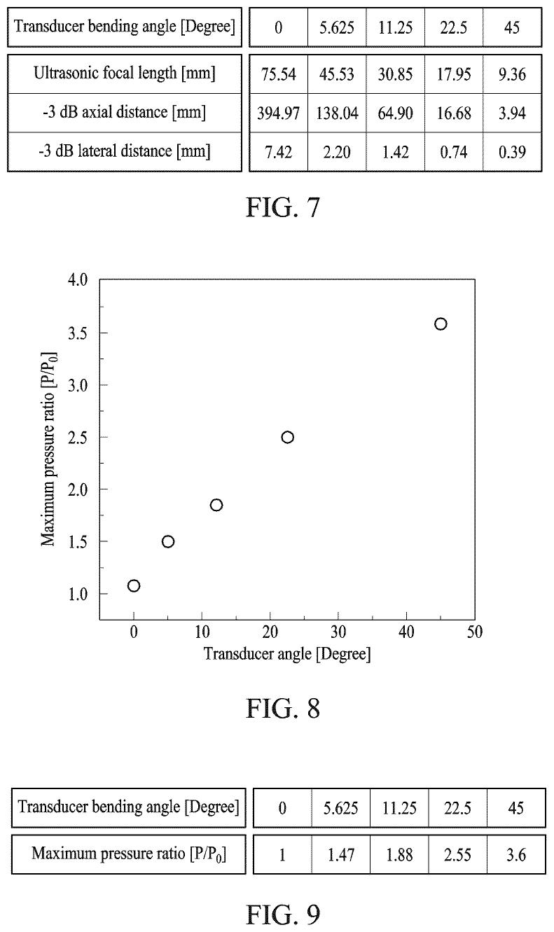

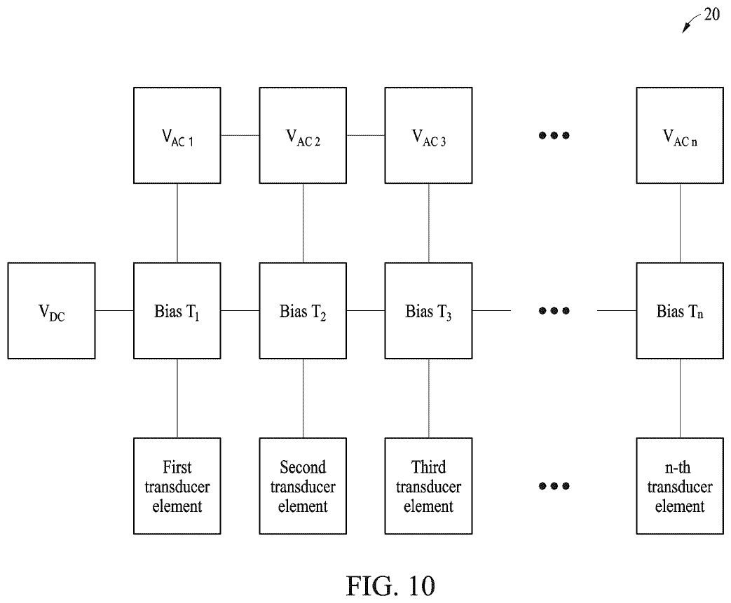

FIG. 6 illustrates graphs of −3 dB distances according to the bending angle of the ultrasonic transducer shown in FIG. 1A, and FIG. 7 is a table illustrating the −3 dB distances according to the bending angle of the ultrasonic transducer shown in FIG. 1A.

Referring to FIGS. 6 and 7, ultrasonic focal lengths, −3 dB axial distances, and −3 dB lateral distances according to bending angles of an ultrasonic transducer including eight transducer elements may be compared.

As the bending angle of the ultrasonic transducer increases, the ultrasonic focal length, the −3 dB axial distance, and the −3 dB lateral distance may decrease. For example, when the bending angle of the ultrasonic transducer is 0°, the focal length may be 75.54 mm, the −3 dB axial distance may be 394.97 mm, and the −3 dB lateral distance may be 7.42 mm. When the bending angle of the ultrasonic transducer is 5.625°, the focal length may be 45.53 mm, the −3 dB axial distance may be 138.04 mm, and the −3 dB lateral distance may be 2.20 mm. When the bending angle of the ultrasonic transducer is 45°, the focal length may be 9.36 mm, the −3 dB axial distance may be 3.94 mm, and the −3 dB lateral distance may be 0.39 mm.

As the bending angle of the ultrasonic transducer increases, the ultrasonic focal length, the −3 dB axial distance, and the −3 dB lateral distance may decrease exponentially. For example, when the bending angle of the ultrasonic transducer is 45°, the ultrasonic focal length may decrease to ⅛, the −3 dB axial distance may decrease to 1/100, and the −3 dB lateral distance may decrease to 1/19 compared to those when the bending angle of the ultrasonic transducer is 0°.

That is, as the ultrasonic transducer bends more, the ultrasonic focal length, the −3 dB axial distance, and the −3 dB lateral distance may decrease more, and the ultrasonic beam may be focused on a more minute area.

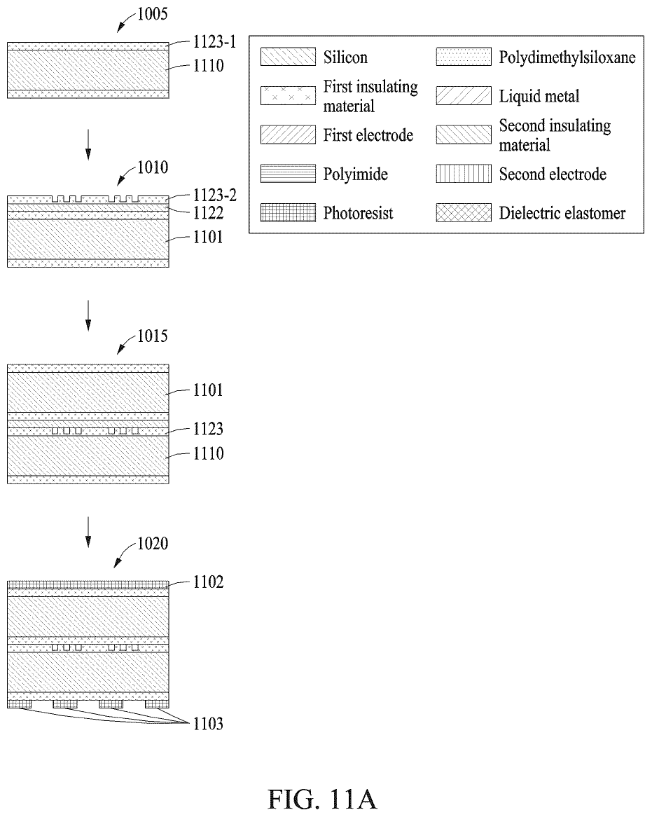

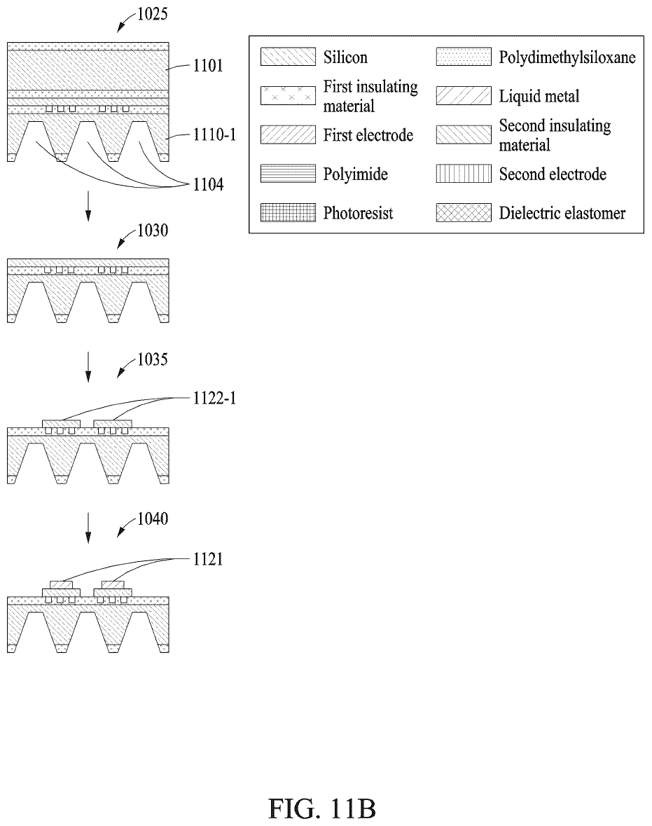

FIG. 8 illustrates a graph of a maximum pressure ratio according to the bending angle of the ultrasonic transducer shown in FIG. 1A, and FIG. 9 is a table illustrating the maximum pressure ratio according to the bending angle of the ultrasonic transducer shown in FIG. 1A.

Referring to FIGS. 8 and 9, maximum pressure ratios (P/P₀) according to bending angles of an ultrasonic transducer including eight transducer elements may be compared.

The maximum pressure ratio (P/P₀) may be calculated through the ratio of the maximum pressure P of a bending ultrasonic transducer to the maximum pressure P₀ of a flat ultrasonic transducer.

As the bending angle of the ultrasonic transducer increases, the maximum pressure ratio may increase. For example, when the bending angle of the ultrasonic transducer is 0°, the maximum pressure ratio may be 1. When the bending angle of the ultrasonic transducer is 11.25°, the maximum pressure ratio may be 1.88. When the bending angle of the ultrasonic transducer is 45°, the maximum pressure ratio may be 3.6.

That is, as the ultrasonic transducer bends more, the maximum pressure ratio may increase more, and the ultrasonic beam may be focused.

Hereinafter, a pitch-catch ultrasonic transducer system will be described in detail.

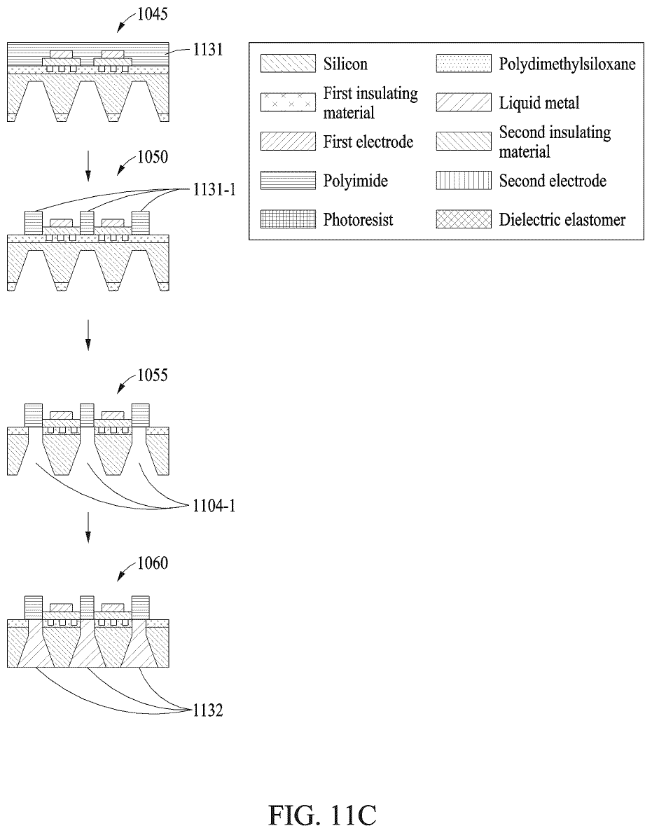

FIG. 10 is a simplified block diagram of another example of an ultrasonic transducer system according to an embodiment.

An ultrasonic transducer system 20 may transmit or receive an ultrasonic wave. The ultrasonic transducer system 20 may connect biases T (e.g., a bias T₁ to a bias Tₙ) respectively to a plurality of transducer elements (e.g., a first transducer element to an n-th transducer element) and separately drive the transducer elements. For example, the ultrasonic transducer system 20 may use the first transducer element as an ultrasonic transmission module and use the n-th transducer element as an ultrasonic reception module. Further, the ultrasonic transducer system 20 may separately drive the second transducer element and the third ultrasonic element to cause various types of ultrasonic beams to be focused.

The ultrasonic transducer system 20 may apply corresponding AC voltages (e.g., V_{AC1} to V_{ACn}) to the respective transducer elements (e.g., the first transducer element to the n-th transducer element), or may apply the same DC voltage V_{DC} to the transducer elements.

Hereinafter, a method of manufacturing the ultrasonic transducer described above will be described in detail.

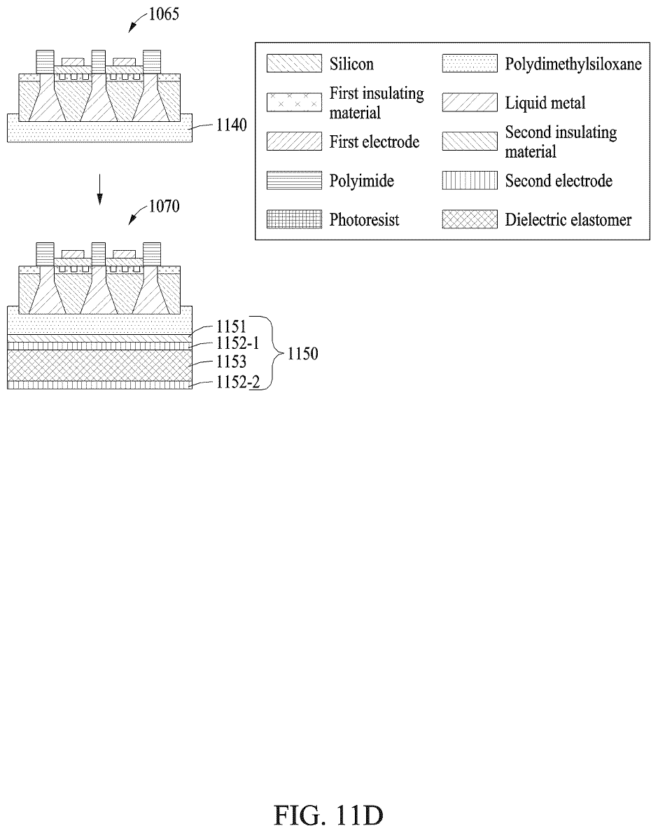

FIGS. 11A to 11D are views illustrating a method of manufacturing the ultrasonic transducer shown in FIG. 1A.

In operation 1005, an oxide film 1123-1 may be formed on a silicon wafer 1110. The oxide film 1123-1 may be formed by furnace equipment that is heat treatment equipment.

In operation 1010, a patterned oxide film 1123-2 may be formed on a membrane layer 1122. The patterned oxide film 1123-2 may be patterned to correspond to a cell design of a transducer element. The patterned oxide film 1123-2 may be formed on a silicon-on-insulator (SOI) wafer 1101 or may be formed on the silicon wafer 1110.

In operation 1015, the silicon wafer 1110 and the SOI wafer 1101 may be joined through bonding. For example, the SOI wafer 1101 may be joined on the silicon wafer 1110 in an inverted state. The oxide film 1123 including the cell design may be formed by joining the oxide film 1123-1 and the patterned oxide film 1123-2 by joining the silicon wafer 1110 and the SOI wafer 1101.

In operation 1020, a photoresist layer 1102 may be formed on the top of the SOI wafer 1101 and a patterned photoresist layer 1103 may be formed on the bottom of the silicon wafer 1110.

In operation 1025, an insulating layer formed on the bottom of the silicon wafer 1110 may be etched based on the patterned photoresist layer 1103, and the silicon wafer 1110 may be etched in an anisotropic form based on the etched insulating layer. A trench 1104 and a substrate 1110-1 may be formed by etching the silicon wafer 1110 using tetramethylammonium hydroxide (TMAH).

In operation 1030, all the layers (e.g., the SOI wafer 1101) positioned on the top of the membrane layer 1122 may be removed through a mechanical method and a chemical method.

In operation 1035, a plurality of membranes 1122-1 may be formed by patterning the membrane layer 1122. The membrane 1122-1 may vibrate to generate an ultrasonic wave.

In operation 1040, a first electrode 1121 may be formed on the top of the membrane 1122-1. The first electrode 1121 may be formed through a vacuum thin film deposition process using sputter equipment or evaporator equipment. The first electrode 1121 may include chrome and gold.

In operation 1045, a polymer layer 1131 may be formed by applying polyimide thereto through a spin coating method.

In operation 1050, a second polymer layer 1131-1 may be formed by patterning the polymer layer 1131.

In operation 1055, a trench 1104-1 may be formed by etching (e.g., wet etching or dry etching) the substrate 1110-1 and the insulating layer 1123 in a radial direction of the trench 1104.

In operation 1060, a liquid metal layer 1132 may be formed by filling the trench 1104-1 with a liquid metal (e.g., a Bi-Pb-In-Sn-Cd fusible alloy).

In operation 1065, a first polymer layer 1140 may be formed on a lower portion of the substrate 1110-1. The first polymer layer 1140 may be formed of polydimethylsiloxane, which is a flexible material, to prevent the fusible alloy of the liquid metal layer 1132 from leaking out when it changes to liquid.

In operation 1070, an actuator layer 1150 may be formed on the bottom of the first polymer layer 1140. An insulating layer 1151 may be formed on the bottom of the first polymer layer 1140 and may be formed of a silicone elastomer material. A first electrode layer 1152-1 may be formed on the bottom of the insulating layer 1151 and may be formed of carbon powder. A dielectric elastomer 1153 may be formed on the bottom of the first electrode layer 1152-1 and may be formed through an acrylic elastomer. A second electrode layer 1152-2 may be formed on the bottom of the dielectric elastomer 1153 and may be formed of carbon powder.

The ultrasonic transducer 100 manufactured by the ultrasonic transducer manufacturing method described above may have a structure of the flexible hinge 130, the polymer layer 140, and the actuator layer 150 and thus bend. Specifically, the ultrasonic transducer 100 may perform a reversible deformation, such as maintaining the shape or deforming, using a phase transition of the liquid metal (e.g., the Bi-Pb-In-Sn-Cd fusible alloy) included in the flexible hinge 130. In addition, the ultrasonic transducer 100 may control the bending angle of the ultrasonic transducer 100 through the dielectric elastomer 153 included in the actuator layer 150, thereby causing the ultrasonic beam to be focused while maintaining the intensity of the ultrasonic beam.

The above-described devices may be configured to act as one or more software modules in order to perform the operations of the above-described embodiments, or vice versa.

A number of embodiments have been described above. Nevertheless, it should be understood that various modifications may be made to these embodiments. For example, suitable results may be achieved if the described techniques are performed in a different order and/or if components in a described system, architecture, device, or circuit are combined in a different manner and/or replaced or supplemented by other components or their equivalents.

Accordingly, other implementations are within the scope of the following claims.

1. An ultrasonic transducer comprising:
a substrate;
a plurality of transducer elements stacked on a top of the substrate to be spaced apart from each other;
a flexible hinge positioned between the plurality of transducer elements and formed to pass through the substrate;
a first polymer layer formed to cover a lower portion of the substrate; and
an actuator layer formed on a bottom of the first polymer layer.

2. The ultrasonic transducer of claim 1, wherein
the flexible hinge comprises:
a second polymer layer positioned over a separation space formed between adjacent transducer elements; and
a liquid metal layer extending from a bottom of the second polymer layer and passing through the substrate.

3. The ultrasonic transducer of claim 1, wherein
the actuator layer comprises:
an insulating layer formed on the bottom of the first polymer layer;
a first electrode layer formed on a bottom of the insulating layer;
a dielectric elastomer formed on a bottom of the first electrode layer; and
a second electrode layer formed on a bottom of the dielectric elastomer.

4. The ultrasonic transducer of claim 1, wherein
the first polymer layer comprises polydimethylsiloxane.

5. The ultrasonic transducer of claim 2, wherein
the second polymer layer comprises polyimide.

6. The ultrasonic transducer of claim 2, wherein
the liquid metal layer comprises a bismuth (Bi)-lead (Pb)-indium (In)-tin (Sn)-cadmium (Cd) fusible alloy.

7. The ultrasonic transducer of claim 2, wherein
the liquid metal layer undergoes a phase transition from solid to liquid based on heat generated by a voltage applied to the substrate.

8. The ultrasonic transducer of claim 3, wherein
the dielectric elastomer bends by a voltage applied to the first electrode layer and the second electrode layer when a fusible alloy included in the flexible hinge is in a liquid state.

9. A method of manufacturing an ultrasonic transducer, the method comprising:
forming a substrate;
stacking a plurality of transducer elements on a top of the substrate to be spaced apart from each other;
forming a flexible hinge between the plurality of transducer elements to pass through the substrate;
forming a first polymer layer to cover a lower portion of the substrate; and
forming an actuator layer on a bottom of the first polymer layer.

10. The method of claim 9, wherein
the forming of the flexible hinge comprises:
forming a second polymer layer over a separation space formed between adjacent transducer elements; and
forming a liquid metal layer extending from a bottom of the second polymer layer and passing through the substrate.

11. The method of claim 10, wherein
the forming of the second polymer layer comprises:
stacking a polymeric material on the substrate and the plurality of transducer elements; and
forming the second polymer layer by patterning the polymeric material.

12. The method of claim 11, wherein the forming of the liquid metal layer comprises:
forming a trench by etching the substrate positioned on the bottom of the second polymer layer; and
forming the liquid metal layer by filling the trench with a liquid metal.

13. The method of claim 9, wherein the forming of the actuator layer comprises:
forming an insulating layer on the bottom of the first polymer layer;
forming a first electrode layer on a bottom of the insulating layer;
forming a dielectric elastomer on a bottom of the first electrode layer; and
forming a second electrode layer on a bottom of the dielectric elastomer.

14. The method of claim 9, wherein the first polymer layer comprises polydimethylsiloxane. The method of claim 10, wherein the stacking of the polymeric material comprises stacking polyimide through spin coating.

16. The method of claim 10, wherein the liquid metal layer comprises a bismuth (Bi)-lead (Pb)-indium (In)-tin (Sn)-cadmium (Cd) fusible alloy.

17. The method of claim 10, wherein the liquid metal layer undergoes a phase transition from solid to liquid based on heat generated by a voltage applied to the substrate. Docket No. 20723.41

18. The method of claim 13, wherein the dielectric elastomer bends by a voltage applied to the first electrode layer and the second electrode layer when a fusible alloy included in the flexible hinge is in a liquid state.

19. An ultrasonic transducer system comprising:
the ultrasonic transducer of claim 1; and
a controller configured to control the ultrasonic transducer.

20. The ultrasonic transducer system of claim 19, wherein the controller is further configured to control a flexible hinge included in the ultrasonic transducer and an actuator layer included in the ultrasonic transducer independently of driving the ultrasonic transducer. 21 Docket No. 20723.41
